(19) United States
(12) Patent Application Publication
(10) Pub. No.: US 2024/0016901 A1
(43) Pub. Date: Jan. 18, 2024

(54) COMPOSITIONS AND METHODS FOR PREVENTING AND TREATING SARS-COV-2 INFECTION

(71) Applicant: The Regents of the University of Michigan, Ann Arbor, MI (US)

(72) Inventor: Jonathan Z. Sexton, Ann Arbor, MI (US)

(21) Appl. No.: 17/927,093

(22) PCT Filed: May 26, 2021
(86) PCT No.: PCT/US2021/034234

§ 371 Date: Nov. 22, 2022

Related U.S. Application Data
(60) Provisional application No. 63/031,310, filed on May 28, 2020.

(51) Int. Cl. A61K 38/40; A61K 31/4706; A61K 31/675; A61P 31/14

(52) U.S. Cl. CPC A61K 38/40; A61K 31/4706; A61K 31/675; A61P 31/14

(57) ABSTRACT

This invention is in the field of medicinal pharmacology. In particular, the present invention relates to pharmaceutical compositions capable of preventing, treating and/or ameliorating symptoms related to conditions caused by the SARS-CoV-2 vims (e.g., COVID-19). The invention further relates to methods of preventing, treating and/or ameliorating symptoms related to conditions caused by the SARS-CoV-2 vims (e.g., COVID-19), comprising administering to a subject (e.g., a human patient) a pharmaceutical composition comprising lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir (alone or with additional agents).

### CROSS-REFERENCE TO RELATED APPLICATION

This application claims priority to U.S. Provisional Patent Application No. 63/031,310 filed May 28, 2020, the contents of which are incorporated by reference in its entirety.

### FIELD OF THE INVENTION

This invention is in the field of medicinal pharmacology. In particular, the present invention relates to pharmaceutical compositions capable of preventing, treating and/or ameliorating symptoms related to conditions caused by the SARS-CoV-2 virus (e.g., COVID-19). The invention further relates to methods of preventing, treating and/or ameliorating symptoms related to conditions caused by the SARS-CoV-2 virus (e.g., COVID-19), comprising administering to a subject (e.g., a human patient) a pharmaceutical composition comprising comprising lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir (alone or with additional agents).

### INTRODUCTION

Severe acute respiratory syndrome coronavirus 2 (SARS-CoV-2) is an enveloped, positive-sense, single-stranded RNA beta-coronavirus that emerged in Wuhan in November 2019 and rapidly developed into a global pandemic. The associated disease, COVID-19, has an array of symptoms, ranging from flu-like illness and gastrointestinal distress (Xiao et al. 2020; Lin et al. 2020) to acute respiratory distress syndrome, heart arrhythmias, strokes, and death (Avula et al. 2020; Kochi et al. 2020). Drug repurposing has played an important role in the search for COVID-19 therapies. Recently, the FDA issued emergency approval of remdesivir (GS-5734), a monophosphoramidate prodrug of a nucleoside inhibitor developed for Ebola virus treatment (Mulangu et al. 2019), and hydroxychloroquine, an aminoquinoline derivative first developed in the 1940s for the treatment of malaria, for severely ill patients with COVID-19. However, there are no established prophylactic strategies or direct antiviral treatments available to limit SARS-CoV-2 infections and to prevent/cure the associated disease COVID-19.

Prophylactic strategies and/or direct antiviral treatments for preventing, treating and ameliorating the symptoms of SARS-CoV-2/COVID-19 are desperately needed.

The present invention addresses this need.

### SUMMARY

Repurposing of FDA-approved drugs is a promising strategy for identifying rapidly deployable treatments for COVID-19. Benefits of repurposing include known safety profiles, robust supply chains, and a short time-frame necessary for development (Oprea et al. 2011). Additionally, approved drugs serve as chemical probes to understand the biology of viral infection and can make new associations between COVID-19 and molecular targets/pathways that influence pathogenesis of the disease. A complementary approach to standard in vitro antiviral assays is high-content imaging-based morphological profiling. Using morphological profiling, it is possible to identify pathways and novel biology underlying infection, thus allowing for targeted screening around a particular biological process or targeting of host processes that limit viral infection. This enables the identification of multiple anti-viral mechanisms, allowing for the rational design of drug combinations or, conversely, revealing drugs that exacerbate infectivity or are associated with cytotoxicity.

Experiments conducted during the course of identifying embodiments for the present invention resulted in the development of a pipeline for quantitative high-throughput image-based screening of SARS-CoV-2 infection. Machine learning approaches were leveraged to create an assay metric that accurately and robustly identifies features that predict antiviral efficacy and mechanism of action. Several FDA-approved drugs and clinical candidates were identified with unique antiviral activity. Such experiments further demonstrated that lactoferrin inhibits viral entry and replication, enhances antiviral host cell response, and potentiates the effects of remdesivir and hydroxychloroquine. Furthermore, such experiments resulted in the identification of currently prescribed drugs that exacerbate viral infectivity. Collectively, the present invention represents evidence that morphological profiling can robustly identify new potential therapeutics against SARS-CoV-2 infection as well as drugs that potentially worsen COVID-19 outcomes.

Accordingly, the present invention relates to pharmaceutical compositions capable of preventing, treating and/or ameliorating symptoms related to conditions caused by the SARS-CoV-2 virus (e.g., COVID-19). The invention further relates to methods of preventing, treating and/or ameliorating symptoms related to conditions caused by the SARS-CoV-2 virus (e.g., COVID-19), comprising administering to a subject (e.g., a human patient) a pharmaceutical composition comprising comprising lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir (alone or with additional agents).

In certain embodiments, the present invention provides methods for administering a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir to a subject (e.g., a human subject) (e.g., a human subject suffering from or at risk of suffering from a condition related to SARS-CoV-2 infection (e.g., COVID-19)) for purposes of treating, preventing and/or ameliorating the symtpoms of a viral infection (e.g., SARS-CoV-2 infection (e.g., COVID-19)).

In such embodiments, the methods are not limited treating, preventing and/or ameliorating the symtpoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virals having MP″ protease activity and/or expression.

In such embodiments, administration of the pharmaceutical composition results in suppression of pro-inflammatory cytokine activity (e.g., IL-6 activity) within the subject. In some embodiments, administration of the pharmaceutical composition results in enhancement of NK cell activity within the subject. In some embodiments, administration of the pharmaceutical composition results in enhancement of neutrophil activity within the subject. In some embodiments, administration of the the pharmaceutical composition results in inhibition of viral entry into the subject's cells through inhibiting binding of the virus with heparin sulfate proteoglycan within such cells.

In some embodiments, the pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir is administered in combination with remdesivir (if not in the pharmaceutical composition) or hydroxychloroquine.

In such embodiments wherein the pharmaceutical composition comprises lactoferrin, the lactoferrin is obtained through isolation and purification from natural sources, for example, but not limited to mammalian milk. The lactoferrin is preferably mammalian lactoferrin, such as bovine or human lactoferrin. In some embodiments, the lactoferrin is human lactoferrin produced recombinantly using genetic engineering techniques well known and used in the art, such as recombinant expression or direct production in genetically altered animals, plants or eukaryotes, or chemical synthesis (see, U.S. Pat. Nos. 5,571,896; 5,571,697 and 5,571,691).

In certain embodiments, the present invention provides methods for treating, ameliorating and/or preventing a condition related to viral infection in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments; the present invention provides methods for treating, ameliorating and/or preventing SARS-CoV-2 infection (e.g., COVID-19) in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition comprising lactoferrin is configured for oral administration. In some embodiments, the subject is a human subject.

In certain embodiments, the present invention provides methods for treating, ameliorating and/or preventing symptoms related to viral infection in a subject, comprising administering to the subject a pharmaceutical composition comprising lactoferrin. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous, topical). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous; topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisterization by nebulization, anti-tussives, respiratory stimulants, surgery and alpha 1 antitrypsin.

In certain embodiments, the present invention provides methods for treating, ameliorating and/or preventing gastrointestinal conditions in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for treating, ameliorating and/or preventing gastrointestinal conditions related to SARS-CoV-2 infection (e.g., COVID-19) in a subject, comprising administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for inhibiting viral entry in a cell, comprising exposing the cell a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising lactoferrin results in suppression of pro-inflammatory cytokine activity (e.g., IL-6 activity) within the cell. In some embodiments, exposure of the cell to the pharmaceutical composition comprising lactoferrin results in enhancement of NK cell activity within the cell. In some embodiments, exposure of the cell to the pharmaceutical composition comprising lactoferrin results in enhancement of neutrophil activity within the cell. In some embodiments, exposure of the cell to the pharmaceutical composition comprising lactoferrin results in inhibition of viral entry into the cell through inhibiting binding of the virus with heparin sulfate proteoglycan within the cell.

In certain embodiments; the present invention provides kits comprising (1) a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir, (2) a container, pack, or dispenser, and (3) instructions for administration. In some embodiments, the kit further comprises remdesivir (if not in the pharmaceutical composition) or hydroxychloroquine.

### BRIEF DESCRIPTION OF THE DRAWINGS

FIG. 1A-C: A) Growth kinetics of Vero E6, Huh-7 and Caco-2 cells. Cells were infected in 48-well plates with SARS-CoV-2 at an MOI of 0.2 and harvested at day 0 (1 h post adsorption), day 1, 2 and 3. TCID50 determination was performed on the supernatant and cellular fraction. Graph represents median, SD of N=2 biological replicates with n=3 technical replicate each. B) Syncytia formation (magenta, anti-SARS-CoV-2 NP antibody) in SARS-CoV-2 infected Vero E6 (left) and Caco2 (right) cells counter stained with Hoechst 33342 (cyan) and anti-SARS-CoV-2 NP antibody (magenta). C) Limit of detection of the assay. Huh-7 cells were infected at indicated MOI and imaged at 48 h post infection (p.i.). Progressive and more feature-rich syncytia formation was observed in correlation with an increased MOI and detection was possible with infection as low as MOI 0.004.

FIG. 2: Morphological profiling of SARS-CoV-2 infected Huh-7 cells (MOI of 0.2 for 48 hrs). Center image: representative field with nuclei (cyan), neutral lipids (green), and SARS-CoV-2 NP protein (magenta). Through feature extraction key traits of SARS-CoV-2 infection were characterized with multinucleated syncytia (top left) and abundant nucleoli (bottom left) from HCS CellMask Orange channel. Cell viral compartmentalization (top right) with cytoplasmic protrusions (bottom right) from SARS-CoV-2 NP channel. Representative image was acquired on a Yokogawa CQ1 high-content imager and analyzed with Fiji ImageJ package.

FIG. 3A-B: A) Dose-response curve of the 132 hits from the qHTS screening. XX B) Replicability plot showing a strong correlation in antiviral efficacy between replicate plates.

FIG. 4A-B: a) Schematic representation of the anti-SARS-CoV-2 therapy discovery effort. 1) Compounds are administered to cells cultured on 384-well plates infected with SARS-CoV-2. Each plate contains 24 negative (infected) and 24 positive (non-infected) control wells to control for plate-to-plate variation. 2) Cells are fixed, stained, and imaged. Images are analyzed through a Cell Profiler-based pipeline which segments nuclei, cell boundaries, neutral lipid content and viral syncytia formation while extracting features of these cellular compartments. 3) Dose-response curves are calculated through multivariate-analysis to define per-image viral infectivity 4) Machine learning models are built around positive and negative control wells based on extracted features and applied to each drug condition. 5) Models inform on individual compound mode(s) of antiviral action through obtained features 6) confirmed antiviral hits; b) Dose-response curves of 15 hits of the drug screening. Graphs represent median SEM of 10-point 1:2 dilution series of selected compounds for N=3 biological replicates. IC50 were calculated based on normalization to the control and after fitting in GraphPad Prism.

FIG. 5A-C: Embedding of cells by their morphological features shows clustering by cellular state and infection status a) 2 dimensional UMAP embedding of two million individual cells by 379 morphological features consisting of uninfected (PC), infected (NC), or infected and treated with 12 FDA approved and clinical candidate drug screening hits across 10 doses. b) Cluster regions of interest (ROI) in the UMAP are highlighted including infected syncytial (ROI 3) and isolated (ROI 4) cells and non-infected mitotic (ROI 6), normal (ROI 10), scattered lipid (ROI 11), and cytoplasm punctate (ROI 12) cells. c) For six ROIs, a representative cell is shown by nuclear (upper-left), cell boundary (upper-right), neutral lipid (lower-left), and SARS-CoV-2 nucleocapsid (lower-right) channels. Below, the cell count across each treatment and dose is shown as a heat-map, where the dose-responsive behavior for ROIs 3 and 4 are visible.

FIG. 6A-H: Lactoferrin blocks SARS-CoV-2 replication at different stages of the viral cycle. a) Huh-7 cells were treated with lactoferrin (0 to 2.3 μM) and infected with SARS-CoV-2 (MOI of 0.2) in a 384-well plate. Plates were imaged using automated fluorescence microscopy and processed using our image analysis pipeline to determine percent viral inhibition. Graph indicates a dose-response (RED, IC50=308 μM). Cell viability is depicted in black. b) Huh-7 were infected with SARS-CoV-2 (MOI of 1, 5 and 10; MOI of 0 indicates non-infected cells) and treated with 2.3 μM of Lactoferrin at 1 and 24 hrs p.i. Bars indicate the percentage of infected cells in different conditions. Data is an average of 8 replicates. Statistical significance determined using multiple T-test with the Bonferroni-Dunn method, with alpha=0.05. Except for MOI of 0, all conditions (Untreated vs Lactoferrin, 1 hr or Untreated vs Lactoferrin, 24 hr) have a p-value <0.0001. c-d) 2.5×10⁴ Huh-7 cells were infected with SARS-CoV-2 at MOI of 0.2. 48 hrs p.i., cells were harvested and RNA was extracted. Viral genome copies were calculated with an absolute quantification method (standard curve) (c) and mRNA levels of cellular IFNβ, MX1, ISG15 and IFITM3 (d) were calculated with ΔΔCt over non-infected Huh-7. Data are average, SD of N=2 biological replicates with n=3 technical replicates each. Statistical significance determined using multiple T-test with the Bonferroni-Dunn method, with alpha=0.05. *p-value <0.001. e) Percentage of SARS-CoV-2 infected Huh-7 cells upon treatment with Apolactoferrin, Hololactoferrin, and Transferrin at a concentration of 2.3 μM. f) Percentage of infected cells 48 hrs p.i. at MOI of 10 upon treatment with remdesivir (50 nM), lactoferrin (1.2 μM), and remdesivir/lactoferrin (25 nM/600 nM) combination treatment. g) and h) 2-dimensional dose response heat maps of lactoferrin in combination with remdesivir and hydroxychloroquine. remdesivir combination was evaluated with a 0.2 MOI and HCQ was evaluated with a MOI of 10 leading to a relative shift in lactoferrin potency.

FIG. 7A-B: A) Dose-response curve for remdesivir alone and in combination with 280 nM and 1 μM lactoferrin showing potentiation of efficacy. B) Dose-response curve for hydroxychloroquine alone and in combination with 280 nM and 1 μM lactoferrin showing potentiation of efficacy.

### DETAILED DESCRIPTION OF THE INVENTION

The global spread of the severe acute respiratory syndrome coronavirus 2 (SARS-CoV-2), and the associated disease COVID-19, requires therapeutic interventions that can be rapidly translated to clinical care. Unfortunately, traditional drug discovery methods have a >90% failure rate and can take 10-15 years from target identification to clinical use. Drug repurposing, i.e., the use of previously developed and tested agents for different disease states, can significantly accelerate translation.

Experiments conducted during the development of embodiments for the present invention resulted in the development of a quantitative high-throughput screen to identify efficacious single agents and combination therapies against SARS-CoV-2. Quantitative high-content morphological profiling was coupled with an AI-based machine learning strategy to classify features of cells as positive or negative for infection and stress. This assay detected multiple mechanisms of anti-viral actions, including inhibition of viral entry, propagation, and modulation of host cellular responses. From a library of 1,441 FDA-approved compounds and clinical candidates, 15 dose-responsive compounds with antiviral effects were identified (e.g., lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir). In particular, it was discovered that lactoferrin is an effective inhibitor of SARS-CoV-2 infection with an IC₅₀ of 308 nM and that it potentiates the efficacy of both remdesivir and hydroxychloroquine. Lactoferrin was also shown to stimulate an antiviral host cell response and retain inhibitory activity in iPSC-derived alveolar epithelial cells. Given its safety profile in humans, these preclinical data indicate that lactoferrin is a readily translatable therapeutic adjunct for Covid-19. Additionally, several commonly prescribed drugs were found to exacerbate viral infection and warrant clinical investigation of worse patient outcomes.

Accordingly, the present invention relates to pharmaceutical compositions capable of preventing, treating and/or ameliorating symptoms related to conditions caused by the SARS-CoV-2 virus (e.g., COVID-19). The invention further relates to methods of preventing, treating and/or ameliorating symptoms related to conditions caused by the SARS-CoV-2 virus (e.g., COVID-19), comprising administering to a subject (e.g., a human patient) a pharmaceutical composition comprising comprising lactoferrin,

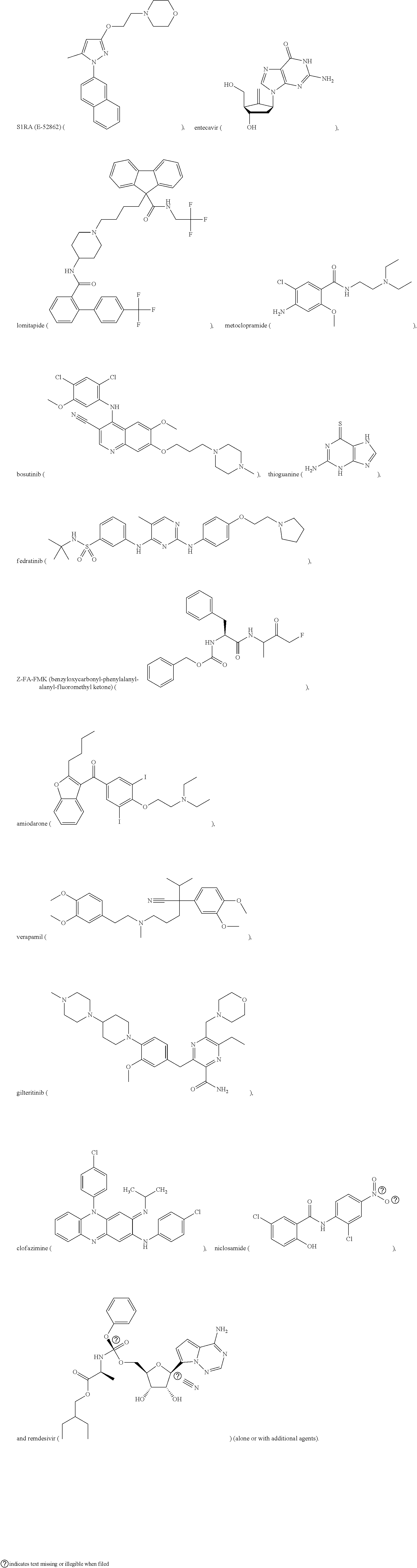

An important aspect of the present invention is that the pharmaceutical compositions comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir are useful in treating viral infection (e.g., SARS-CoV-2 infection) and symptoms related to such a viral infection (e.g., fever, fatigue, dry cough, myalgias, dyspnea, acute respiratory distress syndrome, and pneumonia).

Some embodiments of the present invention provide methods for administering an effective amount of a pharmaceutical composition comprising lactoferrin and at least one additional therapeutic agent (including, but not limited to, any pharmaceutical agent useful in treating SARS-CoV-2 infection and/or symptoms related to such a viral infection (e.g., fever, fatigue, dry cough, myalgias, dyspnea, acute respiratory distress syndrome, and pneumonia). In some embodiments, the additional agent is remdesivir (if not in the pharmaceutical composition) and/or hydroxychloroquine.

Compositions within the scope of this invention include all pharmaceutical compositions contained in an amount that is effective to achieve its intended purpose. While individual needs vary, determination of optimal ranges of effective amounts of each component is within the skill of the art. Typically, the pharmaceutical agents which function as inhibitors of SARS-CoV-2 viral entry may be administered to mammals, e.g. humans, orally at a dose of 0.0025 to 50 mg/kg, or an equivalent amount of the pharmaceutically acceptable salt thereof, per day of the body weight of the mammal being treated. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the inhibiting agent. The unit dose may be administered one or more times daily as one or more tablets or capsules each containing from about 0.1 to about 10 mg, conveniently about 0.25 to 50 mg of the agent (e.g., mimetic peptide, small molecule) or its solvates.

In a topical formulation, the lactoferrin may be present at a concentration of about 0.01 to 100 mg per gram of carrier. In a one embodiment, the lactoferrin is present at a concentration of about 0.07-1.0 mg/ml, for example, about 0.1-0.5 mg/ml, and in one embodiment, about 0.4 mg/ml.

In addition to administering one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir as a raw chemical, any may be administered as part of a pharmaceutical preparation containing suitable pharmaceutically acceptable carriers comprising excipients and auxiliaries which facilitate processing of the lactoferrin into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active mimetic peptide(s), together with the excipient.

The pharmaceutical compositions of the invention may be administered to any patient that may experience the beneficial effects of one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir. Foremost among such patients are mammals, e.g., humans, although the invention is not intended to be so limited. Other patients include veterinary animals (cows, sheep, pigs, horses, dogs, cats and the like).

The pharmaceutical compositions comprising one or more of lactoferrin, S1RA, entecavir, lomitapide, metoclopramide, bosutinib, thioguanine, fedratinib, Z-FA-FMK, amiodarone, verapamil, gilteritinib, clofazimine, niclosamide, and remdesivir may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known; for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Other pharmaceutical preparations that can be used orally include push-fit capsules made of gelatin, as well as soft, sealed capsules made of gelatin and a plasticizer such as glycerol or sorbitol. The push-fit capsules can contain the active mimetic peptides in the form of granules that may be mixed with fillers such as lactose, binders such as starches, and/or lubricants such as talc or magnesium stearate and, optionally, stabilizers. In soft capsules, the active mimetic peptides are in one embodiment dissolved or suspended in suitable liquids, such as fatty oils, or liquid paraffin. In addition, stabilizers may be added.

Possible pharmaceutical preparations that can be used rectally include, for example, suppositories, which consist of a combination of one or more of the active mimetic peptides with a suppository base. Suitable suppository bases are, for example, natural or synthetic triglycerides, or paraffin hydrocarbons. In addition, it is also possible to use gelatin rectal capsules that consist of a combination of the active mimetic peptides with a base. Possible base materials include, for example, liquid triglycerides, polyethylene glycols, or paraffin hydrocarbons.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than Cu). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### Experimental

Morphological Profiling Reveals Unique Features Associated with SARS-CoV-2 Infection

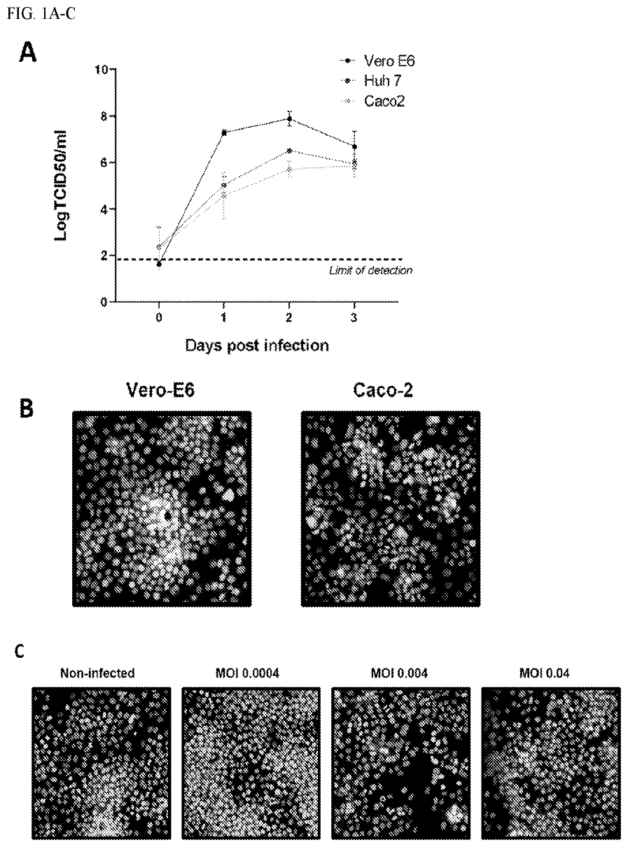

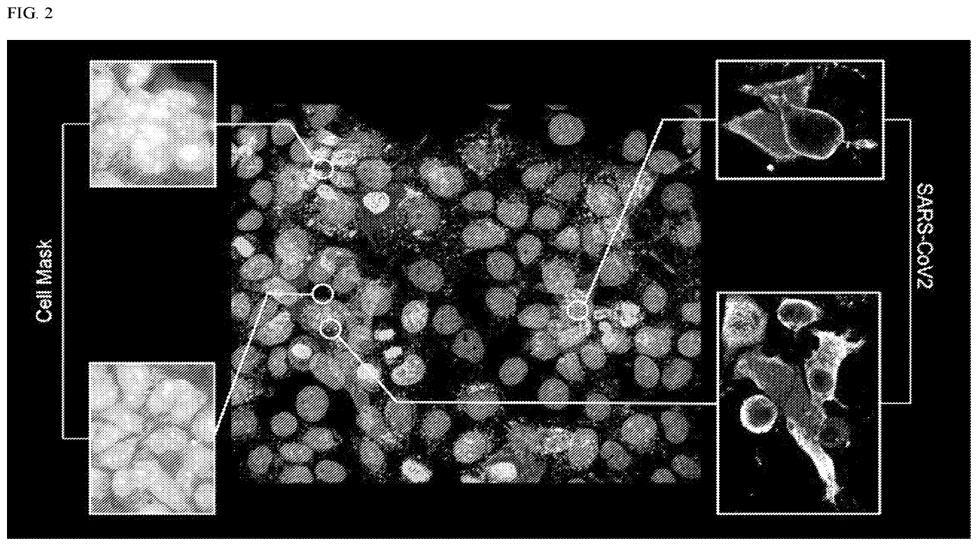

To determine the optimal cell line and appropriate endpoint for antiviral drug screening, experiments were conducted that assessed SARS-CoV-2 infectivity in previously reported permissive cell lines: Vero-E6, Caco-2, and Huh-7 (Chu et al. 2020). Viral growth kinetics at a multiplicity of infection (MOI) of 0.2 revealed that Vero-E6, Caco2, and Huh-7 cells supported viral infection, with peak viral titers at 48 hours post infection (hrs p.i.) (FIG. 1A/B). Although the viral load was higher in Vero-E6 cells, Huh-7 was selected for a morphological drug screen because Huh-7 is a human cell line that expresses both ACE2 and TMPRSS2, which are the primary entry factors for SARS-CoV-2 (Hoffmann et al. 2020). Interestingly, Huh-7 cells supported detectable infection at an MOI as low as 0.004 at 48 hrs p.i. (FIG. 1C) which highlights the high sensitivity of image-based screening. To identify compounds that inhibit or exacerbate infection, an MOI of 0.2 was selected, leading to a baseline infectivity rate of 20%.

Cell-level morphological profiling was enabled through multiplexed staining and automated high-content fluorescence microscopy. The multiplexed dye set included markers for SARS-CoV-2 nucleoprotein, nuclei (Hoechst 33342), neutral lipids (HCS LipidTox Green), and cell boundaries (HCS CellMask Orange). These fluorescent probes were chosen to capture a wide variety of cellular features relevant to viral infectivity, including nuclear morphology, nuclear texture, cytoplasmic and cytoskeletal features, and indicators of cell health and function. From our initial profiling three prominent morphological features associated with SARS-CoV-2 infection were observed: formation of syncytia, increased nucleoli count, formation of cytoplasmic protrusions (FIG. 2). These features, which are key indicators of SARS-CoV-2 infection, were used to generate a machine learning pipeline for antiviral drug screening.

Machine Learning Identifies FDA-Approved Molecules with Antiviral Activity Against SARS-CoV-2

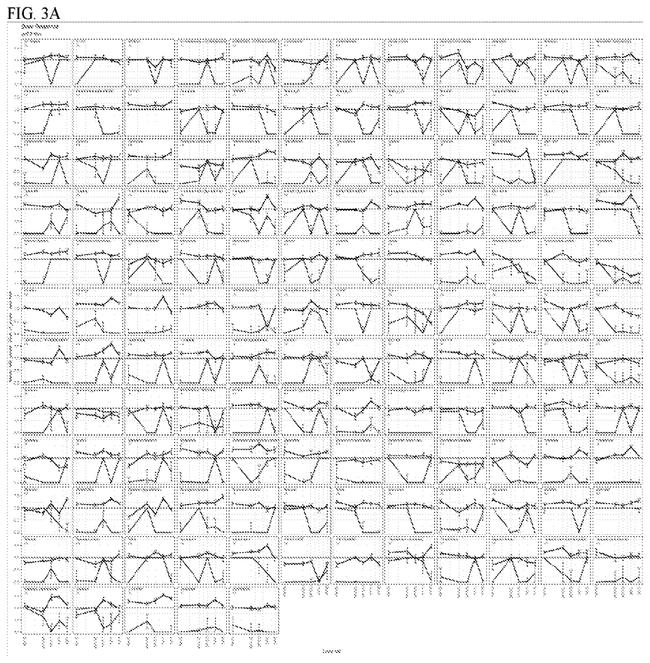

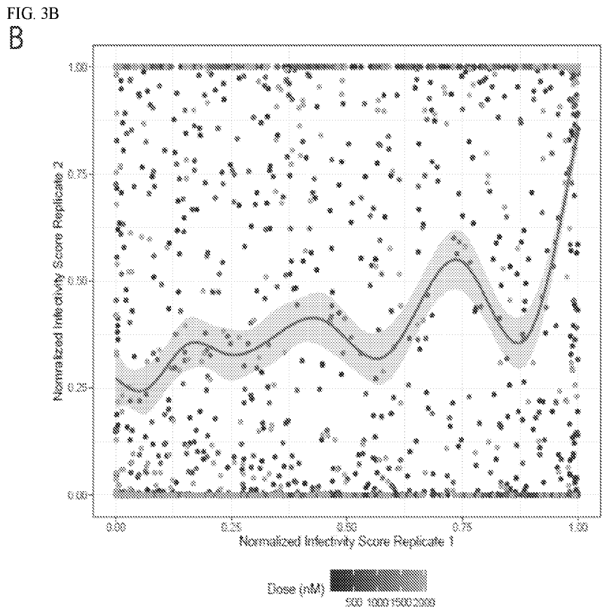

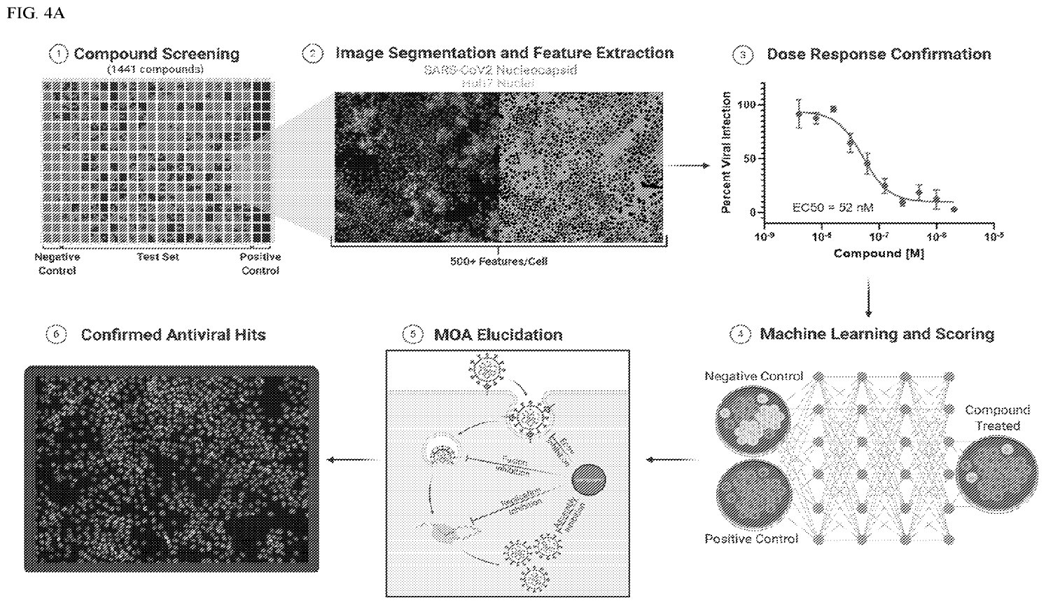

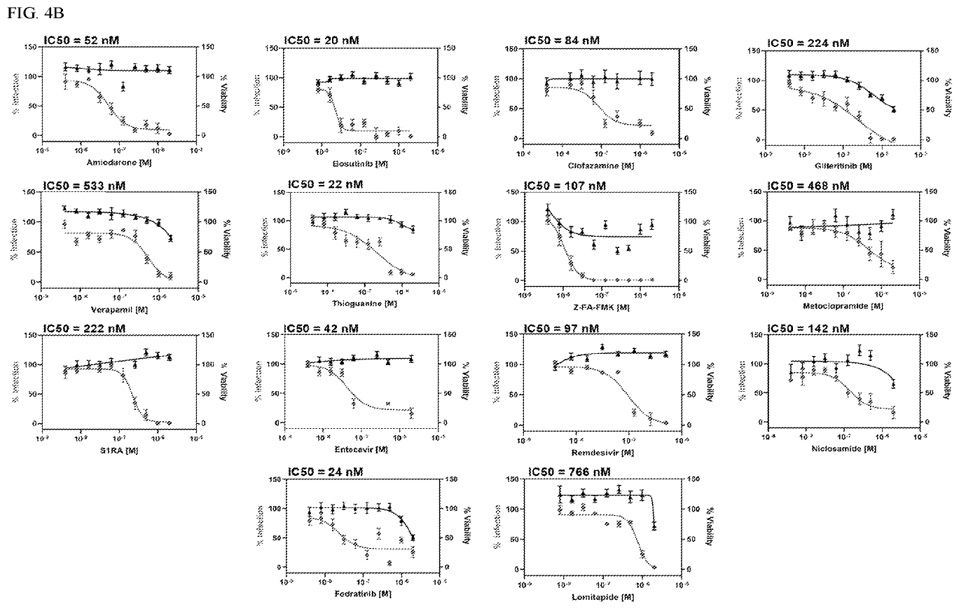

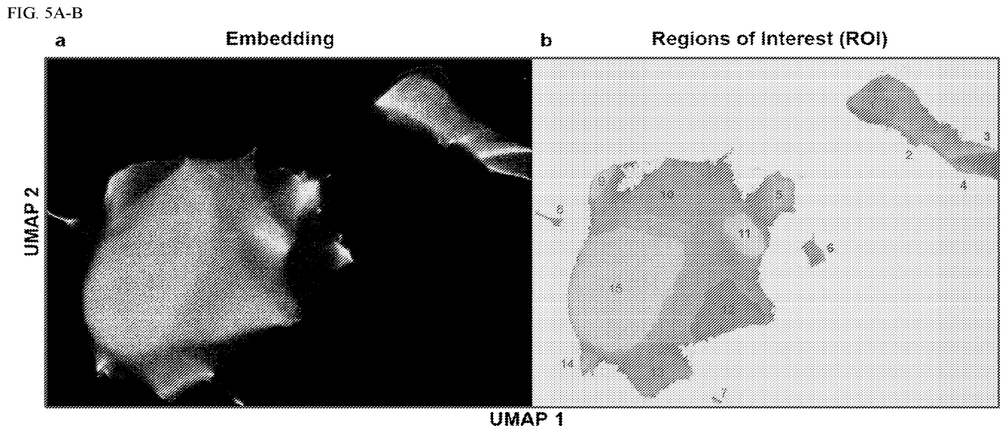

To identify compounds with antiviral activity against SARS-CoV-2, a custom library of 1,441 FDA-approved compounds and rationally included clinical candidates were screened in Huh-7 cells. A first-pass quantitative high-throughput screening (qHTS) approach identified 132 compounds with moderate dose-responsive antiviral behavior between 50 nM and 2 and successfully eliminated compounds without any significant activity or resulted in harsh decrease in cell count (FIG. 3A). The library of compounds were assessed efficiently for their antiviral activity using a CellProfiler-based image analysis and a follow up random forest classification algorithm to identify infected cells and quantify their morphological characteristics (FIG. 4A). This random forest classifier leveraged the quantification of 660 unique cellular features including measurements of intensity, texture and radial distribution for each fluorescent channel (nuclei, cytoplasm, lipid, virus). From the primary qHTS screen, hits were defined as compounds with consistent decreases in viral infectivity in at least three of the tested concentrations as well as minimal cytotoxicity. This approach for hit identification was intentionally designed to be broad in order to minimize false negatives and maximize our list of efficacious compounds which would later be refined. To validate the reproducibility of the primary qHTS screen, a subset of compounds (320) was chosen to perform a biological replicate study that showed correlation with the original screen (FIG. 3B).

Follow-up 10-point, two-fold dilution dose-response experiments were performed in triplicate on the preliminary 132 qHTS hits and validated dose-responsive efficacy for 15 compounds (Table 1 and FIG. 4B). These hits include nine that are novel in vitro observations (lactoferrin, S1RA, entecavir, lomitapide, metoclopramide, bosutinib, thioguanine, fedratinib, Z-FA-FMK), and six which have been previously identified to have antiviral activity (amiodarone, verapamil, gilteritinib, clofazimine (Riva et al. 2020; Heiser et al. 2020), niclosamide (Jeon et al.), and remdesivir) and demonstrate viability of our screening results. In addition to antiviral drug hits, several compounds were also identified that appear to exacerbate SARS-CoV-2 infection in Huh-7 cells including anastrozole, an anti-estrogen drug used to treat breast cancer, and Carbidopa, used to treat Parkinson's disease (Table 3).

TABLE 1

| ID Drug | Compound | | General mechanism | Potential mode of action | EC50 |
|---|---|---|---|---|---|
| Bank ID | name | Current clinical indication | of action | against SARS-COV2 | (nM) |
| DB01118 | Amiodarone | Treatment of ventricular | Inhibitor of K and | Inhibits SARS | 52 |
| | (hydrochloride) | tachycardia | Ca channels | coronavirus infection | |
| | | | | at a post-endosomal level | |
| | | | | Evaluated in clinical trial for | |
| | | | | COVID19 (NCT04351763) | |
| DB06616 | Bosutinib | Treatment of chronic myelo id | Bcr-Abl kinase inhibitor | The related molecule Imatinib | 28 |
| | | leukemia | | blocks fusion of S protein | |
| DB00845 | Clofazamine | Treatment of leprosy | Bind to mycobacterial | Decrease in lipid accumulation, | 105 |
| | | | DNA and | increase in lipolysis, | |
| | | | | and activation | |
| | | | K+ transporters | of innate immunity | |
| DB00442 | Entecavir | Treatment of hepatitis B virus | Guanine analogue inhibits | Nucleoside inhibitor? | 201 |
| | (hydrate) | | viral replication | | |
| DB12500 | Fedratinib | Treatment of intermediate-2 and | Tyrosine kinase | Predicted inhibition of kinase | 25 |
| | | high risk primary and secondary | inhibitor (Jak2) | (NAK) family—including | |
| | | myelofibrosis | | AAK1 and GAK—reported to | |
| | | | | reduce viral infection in vitro | |
| | | | | Reduction of TH17 responses | |
| | | | | resposible of SARS-COV2 | |
| | | | | associated cytokine storm | |
| DB12141 | Gilteritinib | Treatment of | FMS-like tyrosine | Reported to have | 207 |
| | | FLT3-mutated acute | kinase 3 | antiviral activity | |
| | | myeloid leukemia (AML) | (FLT3) inhibitor | in vitro vs SARS-COV2 | |
| | Lacto ferrin | Diet supplement | Immune-modulator | Entry and post-entry inhibitor | 308 |
| | | | Pan-antiviral activity | | |
| DB08827 | Lomitapide | Treatment of | Microsomal | Lipid metabolism | 1575 |
| | | homozygous familial | triglyceride transfer | | |
| | | hypercholesterolemia | protein (MTP) inhibitor | | |
| DB01233 | Metoclopramide | Treatment of diabetic | Dopamine D2 and serotonin | Dopamine receptor and other | 469 |
| | | gastroparesis | 5-HT3 receptor inhibitor | channels | |
| DB06803 | Niclosamide | Treatment of tapeworm | DNA binding | | 142 |
| | | infections | | | |
| DB14761 | Remdesivir | Investigational for Ebola virus | Nucleoside inhibitor | SARS-COV2 inhbitor | 95 |
| | | treatment | | Evaluated in four clinical | |
| | | | | trials for COVID19 | |
| ZINC95000617 | S1RA | Treatment of neuropathic pain | Sigma R1/R2 modulator | | 222 |
| | | (phase II) | | | |
| DB00352 | Thioguanine | Therapy of acute leukemia | DNA binding | Suppression of Rac1 | 232 |
| | | | | GDP/GTP cycling | |
| DB00661 | Verapamil | Treatment of high | Calcium channel blocker | Evaluated in clinical trial for | 458 |
| | (hydrochloride) | blood pressure, | | COVID19 (NCT04351763) | |
| | | heart arrhythmias, and anginT | | | |
| CAS | Z-FA-FMK | | Irreversible inhibitor of | Cathepsine L inhibitor | 107 |
| 197855-65-5 | | | cysteine proteases | | |

### Cell Level Feature Clustering Reveals Potential Mechanisms of Action for Lead Compounds

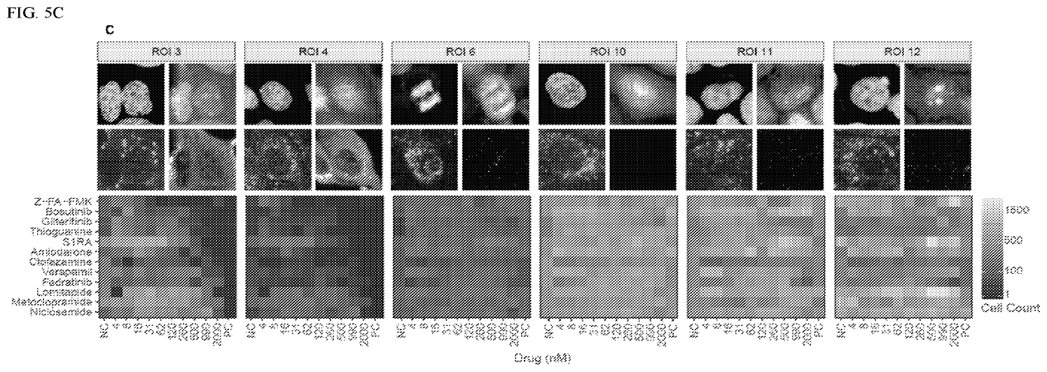

In contrast to standard in vitro assays with a single output, morphological profiling allows for the efficient visualization and quantification of unique biological characteristics of viral infection and cytotoxicity. This facilitates the identification of potential mechanisms of action for antiviral compounds through measured host-cell perturbations upon treatment. Thus, even for previously identified hits, antiviral mechanisms were able to be characterized that cannot be captured through traditional screening assays. For example, entry inhibitors could be identified by a complete lack of signal in the virus channel. In contrast, replication inhibitors often resulted in perinuclear spots in the viral channel: it was hypothesized that these punctae are the remnants of an abortive infection. To better analyze the hits for potential mechanisms of action across the 660 measured cellular features, dimensionality reduction using the uniform manifold approximation and projection (UMAP) embedding was used to project the cellular feature vector into a 2-dimensional plot to observe clusters of cells based on their distinct morphological features (McInnes et al. 2018) (FIG. 5A).

15 regions of interest (ROI) were identified with high cell density (FIG. 5B). A broad density (ROI 9,10) contained uninfected cells with satellite populations having characteristic morphologies including cell division (ROI 6), scattered lipids (ROI 11), and punctate cytoplasm (ROI 12). A large disconnected density (ROI 1-4) contains individually infected cells (ROI 4), infected cells in syncytia (ROI 4), and cells adjacent to infected cells (ROI 1,2) (FIG. 5C). For each ROI the number of cells across each compound treatment dose was counted. For antiviral hits, dose-responsive decreases in cell counts were observed only in regions with infected cells (ROI 1-4). Additionally, the infected cell population were isolated and excluded features measured from the viral nucleocapsid staining image before re-embedding into the UMAP coordinate space and observed that the infected cells reside in the main cluster body with a homogenous density distribution, indicating that there is no substantial bias in the susceptibility to viral infection for a specific cell phenotype/cluster within Huh-7 cells.

### Lactoferrin Blocks SARS-CoV-2 Replication at Different Stages of the Viral Cycle

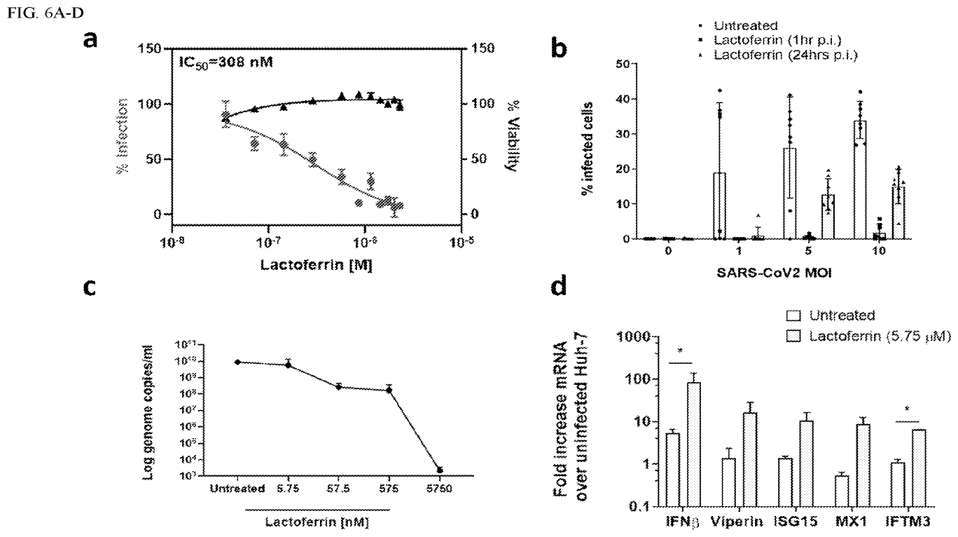

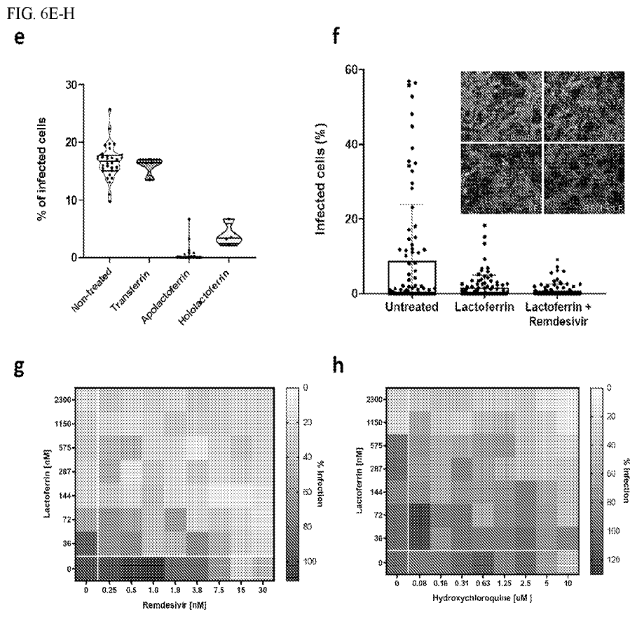

One of the most efficacious hits identified from the morphological screen was lactoferrin, a protein found in milk and other secretory fluids (Lang et al. 2011). It was determined that lactoferrin has dose-dependent (3 nM-2.3 μM) and MOI-dependent (0.2-10) antiviral activity (FIGS. 6A and B). Previous work on lactoferrin in the context of infection with the related SARS-CoV-1 suggests that it blocks viral entry by binding heparan sulfate proteoglycans that are important for early viral attachment (Lang et al. 2011). These studies showed that lactoferrin blocks SARS-CoV-2 infection at the level of entry and with additional mechanisms of action, as it retains antiviral activity when added 1 or 24 hrs p.i. (FIG. 6B). Lactoferrin has been proposed to modulate innate interferon responses to limit viral replication within host cells (Siqueiros-Cendon et al. 2014). Upon treatment, a dose-dependent reduction of viral replication was observed (FIG. 6C), which was consistent with elevated IFNβ and interferon-stimulated genes ISG15, MX1, Viperin and IFITM3 in lactoferrin-treated Huh-7 cells (FIG. 6D). Interestingly, a robust antiviral effect was detected by both holo and apolactoferrin, the latter being the component of widely available dietary supplements. To rule out a mode of action that involved a general iron depletion mechanism, the protein transferrin was found and it was found that it was devoid of any anti-SARS-CoV-2 activity at a concentration of 2.3 μM (FIG. 6E). Lastly, the efficacy of lactoferrin to inhibit SARS-CoV-2 infection in physiologically relevant induced alveolar epithelial cell type II (iAECs) was tested (Jacob et al. 2017; Jacob et al. 2019; Hurley et al. 2020). Consistent with our findings in Huh-7 cells, pretreatment of iAECs with lactoferrin (1.15 μM) resulted in a two-fold reduction in the proportion of SARS-CoV-2 infected cells at MOI 10 (FIG. 6F).

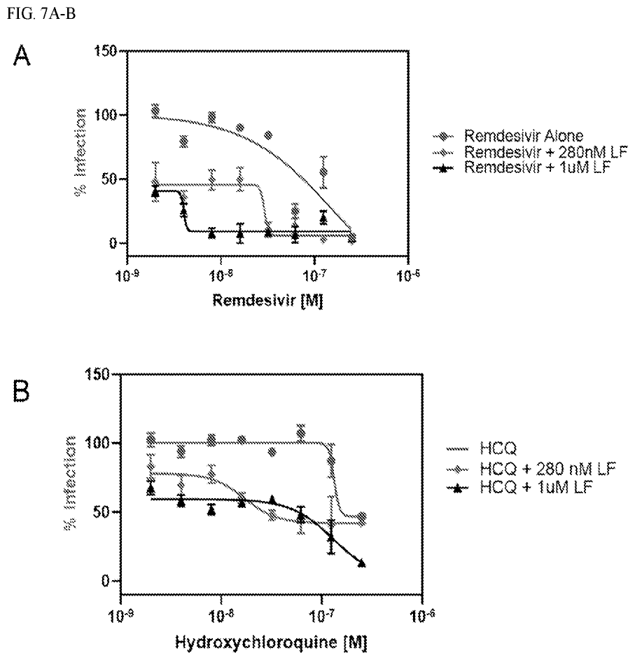

A clinically effective strategy for antiviral therapies uses a combinatorial (or “drug cocktail”) approach, where compounds with varying mechanisms of action are concomitantly used to target different stages in the viral life cycle and to minimize the risk of acquired drug resistance from single-agent selective pressure. This is especially true for RNA viruses, which are highly variable and can develop drug-resistance rapidly (Pallela et al.). Given the high single-agent efficacy of lactoferrin, whether combinations with hydroxychloroquine or remdesivir could improve the overall antiviral activity was tested. It was found that lactoferrin potentiates the efficacy of both remdesivir (FIG. 6G) and hydroxychloroquine (FIG. 6H), which are currently explored treatments for SARS-CoV-2 infection (FIG. 7). Combination therapy with lactoferrin could be beneficial in the management of the COVID-19 pandemic by reducing toxicity (e.g., hydroxycholorquine) or consumption (e.g., remdesivir).

### DISCUSSION

In the experiments conducted during the course of developing embodiments for the present invention, an experimental workflow was developed based on high-content imaging and morphological profiling that allows for rapid screening of FDA-approved compounds, leveraging machine learning to determine potential mechanisms of action. 15 FDA-approved compounds were identified that limit SARS-CoV-2 infection in vitro. Of these, six were previously reported and serve as a benchmark validation of our endpoints and experimental approach and nine were hitherto unknown. This approach was demonstrated as versatile (i.e., it can be applied to both transformed and more physiologically-relevant non-transformed cell lines) and can identify the emergent properties of the infection as well as novel phenotypes that can be perturbed through chemical inhibition.

A high-content morphological profiling approach is superior to image cytometry (tabulating percent positive) and plate reader assays for selecting and prioritizing drugs for repurposing. Viral staining is not merely an absolute measure for viral infection (or inhibition) but the starting point for a detailed investigation of infection trajectories and observations of numerous phenotypic targets, including inhibition of syncytia formation, viral entry, or viral replication, and modulation of the host cell. In contrast to other drug repurposing screens that used a variety of assay technologies and cell models including the Scripps/Reframedb (Riva et al. 2020), Institut Pasteur Korea (Jeon et al.) and Recursion Pharma (Heiser et al. 2020) studies, we report not only compounds with bona fide antiviral activity against SARS-CoV-2 but also their relative mechanism of action.

UMAP visualization is an important advancement in the investigation of cellular phenotypes from cell painting style assays (Bray et al. 2016) and a strategy to characterize pharmacologic perturbations. In the experiments described herein, the UMAP embedding takes the 660 measurements per cell, which comprise the feature vector, and projects them down into a two-dimensional graph for visualization of natural clustering of phenotypes. This non-linear data reduction technique excels at identifying natural phenotypic clusters. In the repurposing screen, it was highly effective at identifying the virally infected cell population and the progression of the viral infection trajectory inside a well was clearly visible. In the uninfected state, cells project to the main cluster body reflecting normal cellular phenotypes. In the Huh7 cell line, there are prominent differences in lipid accumulation and nuclear size/shape/texture, and approximately 10% of the cells are undergoing cell division. These biological processes are clearly visible through inspection of individual cells within cluster areas. In FIG. 5, ROI 6 cells are mitotically active, and a faint connection between the main cluster body and ROI 6 is visible, representing a cell-cycle trajectory that begins in the main cluster body, progresses to ROI 6, and returns to the main cluster body. Similarly, pseudotime can be observed in the context of the viral infection process where cells begin in the main cluster body, traverse to the distant north-east cluster body (ROI 1-4), where it is possible to observe a progression of viral infection starting with a punctate viral signal, progressing to isolated infected cells, and ending with infection of surrounding cells and the formation of syncytia (ROI 3). The UMAP analysis was used to effectively characterize efficacy (reduced density in ROI 1-4) as well as the pharmacologic perturbation of the main cell body. For example, there have been numerous reports of SARS-CoV-2 efficacy with cytotoxic/cytostatic drugs. When these drug-treated wells are embedded in the UMAP coordinate frame, the mitotic cluster disappears, and major shifts in the main cluster body are present along with reduction in the infected cell cluster. This indicates that the uninfected cells are significantly perturbed and the cause of viral replication inhibition is due to global shutdown of transcription/translation. This demonstrates that UMAP embedding can aid in prioritizing efficacious compounds with minimal perturbation in the uninfected population, as observed with remdesivir.

Importantly, the experiments described herein identified drugs that implicate new molecular targets/pathways in the pathogenesis of SARS-CoV-2 and produce clinically-testable and readily translatable hypotheses. As an example, a dose dependent antiviral activity of metoclopramide was observed, a potent D2 receptor antagonist used to treat gastroesophageal reflux disease and prevent other gastrointestinal symptoms, including nausea and vomiting (Hibbs and Lorch 2006). Gastrointestinal symptoms have been increasingly reported in more than half of the patients infected by SARS-CoV-2 (Lin et al. 2020). Notably, investigational drugs like hydroxychloroquine, lopinavir-ritonavir, tocilizumab and others can be associated with gastrointestinal and hepatic adverse events and hence are not ideal for patients already experiencing severe GI symptoms (Hajifathalian et al. 2020). Metoclopramide therefore represents an interesting dual-target therapeutic option for COVID-19 patients.

As most FDA-approved drugs target human molecular targets, the screen helped identify crucial host factors involved in SARS-CoV-2 infection. Z-FA-FMK, an irreversible inhibitor of cysteine proteases, including cathepsins B, L, and S (Roscow et al. 2018), exhibited potent antiviral activity. A recent report using pseudovirus indicated cathepsin L as a crucial entry factor of SARS-CoV-2 (Ou et al. 2020). The antiviral effect of Z-FA-FMK suggests that cathepsin L is a requirement also in the context of SARS-CoV-2 infection and suggests that this molecule could be a useful investigational tool to study virus entry. Similarly, fedratinib is an orally bioavailable semi-selective JAK2 inhibitor, approved by the FDA in 2019 for myeloproliferative neoplasm, a rare blood cancer that causes clotting and fibrosis (Pardanani et al. 2007). JAK-inhibitors have been proposed for COVID-19 to specifically inhibit TH17 mediated inflammatory responses (Wu and Yang 2020; Zhang et al. 2020). In addition, they have been proposed to block numb-associated kinase (NAK) responsible for clathrin-mediated viral endocytosis (Stebbing et al. 2020). Several JAK-inhibitors are currently evaluated in clinical trials for COVID-19 management, including with baricitinib (Treatment of Moderate to Severe Coron . . . ), jakotinib (ChiCTR2000030170), and ruxolitinib (ChiCTR2000029580). Of the tested four FDA approved JAK-inhibitors: baricitinib, ruxolitinib, tofacitinib, and fedratinib, the latter was the only one active against SARS-CoV-2, with an IC₅₀ of 25 nM. However there is some concern that inhibiting the JAK-STAT pathway may limit the protective interferon response (Favalli et al. 2020).

The sigma receptors (SigmaR1/R2) are permissive chaperones that mediate endoplasmic reticulum stress response and lipid homeostasis (Delprat et al. 2020), processes that have been implicated in early stages of hepatitis C viral infection in Huh-7 cells (Friesland et al. 2013) and coronavirus pathogenesis (Fung and Liu 2014). Two sigma receptor modulators were identified: amiodarone (SigmaR1 IC₅₀: 1.4 nM, SigmaR2 IC₅₀: 1 nM) (Moebius et al. 1997), and S1RA (E-52862; SigmaR1 IC₅₀: 17 nM antagonist, SigmaR2 IC₅₀: >1 μM) (Diaz et al. 2012) with potent antiviral activity, demonstrating IC₅₀ values of 52 nM and 222 nM, respectively, with limited cell toxicity. Amiodarone is approved for treatment of arrhythmias but, similar to hydroxychloroquine, potent cardiotoxic side effects through inhibition of the hERG ion channel (Torres et al. 1986) that limit its therapeutic potential. S1RA has completed phase II clinical trials for the treatment of neuropathic pain (Vidal-Torres et al. 2014; Gris et al. 2016). While Gordon et al. identified several other sigmaR1/R2 modulators that inhibited SARS-CoV-2 infection in Vero-E6 cells, antiviral activity for S1RA was not observed (Gordon et al. 2020). This suggests that the activity of S1RA is dependent on host cell factors specific to each cell line and promisingly, that human cells may be more responsive to this compound.

Most noteworthy, the screen demonstrates lactoferrin as a SARS-CoV-2 inhibitor in vitro with multimodal efficacy. Efficacy was showed in multiple cell types, including a non-transformed and clinically relevant iPSC-derived model of airway epithelium. Lactoferrin gene expression has been shown previously to be highly upregulated in response to SARS-CoV-1 infection (Reghunathan et al. 2005) and, in addition to enhancing natural killer cell and neutrophil activity, lactoferrin blocks viral entry through binding to heparan sulfate proteoglycans. Interestingly, lactoferrin retains anti-SARS-CoV-2 activity up to 24 hrs p.i., which suggests additional mechanisms of action other than simple entry inhibition. Although not dependent upon a definitive and complete mechanism of action, the described results showed significant host cell modulation through increased expression of several interferon-stimulated genes upon treatment with lactoferrin. Additionally, lactoferrin has been previously shown to decrease the production of IL-6 (Cutone et al. 2014), which is one of the key players of the “cytokine storm” produced by SARS-CoV-2 infection (Conti et al. 2020; Lagunas-Rangel and Chavez-Valencia 2020). Importantly, we found that lactoferrin retains activity in both the holo and apo forms, the latter being the component of orally available lactoferrin supplements. Orally available lactoferrin could be especially effective in mitigating the gastrointestinal symptoms that are present in COVID-19 patients (Han et al. 2020). The mechanisms may be similar to how lactoferrin reduces human norovirus infection through induction of innate immune responses (Oda et al. 2020), especially as lactoferrin gene polymorphisms are associated with increased susceptibility to infectious diarrhea (Mohamed et al. 2007). If lactoferrin reduces viral load in the GI tract, it could reduce fecal-oral transmission of COVID-19 (Gu et al. 2020).

Combination therapies are likely to be required for effectively treating SARS-CoV-2 infection, and this approach has already shown some promise. For example, combination therapy with interferon beta-1b, lopinavir-ritonavir, and ribavirin showed efficacy against SARS-CoV-2 in a prospective, open-label, randomised, phase 2 trial (Hung et al. 2020). The results described herein demonstrated that lactoferrin potentiates the antiviral activity of both remdesivir and hydroxychloroquine and could be used as a combination therapy with these drugs, which are currently being used or studied for the treatment of COVID-19. Due to its wide availability, limited cost and lack of adverse effects, lactoferrin could be a rapidly deployable option for both prophylaxis and the management of COVID-19.

### Cells and Virus

Vero E6, Caco2 and Huh7 cells were maintained at 37° C. with 5% CO2 in Dulbecco's Modified Eagle's Medium (DMEM; Welgene), supplemented with 10% heat-inactivated fetal bovine serum (FBS), HEPES, non-essential amino-acids, L-glutamine and 1×Antibiotic-Antimycotic solution (Gibco). The iPSC-derived airway epithelial cell line (iAEC) was cultured based on a previously described differentiation process (Hurley et al 2020). In brief, iPSCs are differentiated to NKX2.1 positive lung endoderm in two-dimensional cell culture. NKX2.1 positive cells were flow sorted and embedded in Matrigel (Corning) and cultured in “CK+DCI+Y” media to promote alveolar differentiation. SARS-CoV-2 WA1 strain was obtained by BEI resources and was propagated in Vero E6 cells. Viral titers were determined by TCID50 assays in Vero E6 cells (Reed and Muench method) by microscopic scoring. All experiments using SARS-CoV-2 were performed at the University of Michigan under Biosafety Level 3 (BSL3) protocols in compliance with containment procedures in laboratories approved for use by the University of Michigan Institutional Biosafety Committee (IBC) and Environment, Health and Safety (EHS).

### Viral Quantification: Growth Kinetics and RT-qPCR Assay

Vero E6, Caco2 and Huh7 cells were seeded in a 48-well plate at 2×10\hat{ }4 cells/well incubated overnight at 37° C. with 5% CO2. Cells were then infected with SARS-CoV-2 WA1 at a multiplicity of infection (MOI) of 0.2. One hour after infection, cells were harvested (day 0 of infection) or kept at 37° C. for 1, 2 and 3 days p.i. Viral titer determination was performed by TCID50 assay on Vero E6 cells of the total virus (supernatant and intracellular fraction). Alternatively, cells were harvested with Trizol and total cellular and viral RNA was extracted with the ZymoGen Direct-zol RNA extraction kit. Viral RNA was quantified by RT-qPCR using the 2019-nCoV CDC qPCR Probe Assay and the probe set N1 (IDT technologies). IFNβ, viperin, MX1, ISG15, IFITM3 and the housekeeping gene GAPDH mRNA levels were quantified by qPCR with SsoAdvanced™ Universal SYBR® Green Supermix (Bio-Rad) with specific primers (IFNβ: F-TTGACATCCCTGAGGAGATTAAGC (SEQ ID NO: 1), R-TCCCACGTACTCCAACTTCCA (SEQ ID NO: 2); MX1: F-CCAGCTGCTGCATCCCACCC (SEQ ID NO: 3), R-AGGGGCGCACCTT CTCCTCA (SEQ ID NO: 4); ISG15: F-TGGCGGGCAACGAATT (SEQ ID NO: 5), R-GGGTGATCTGCGCCTTCA (SEQ ID NO: 6); IFITM3: F-TCCCAC GTACTCCAACTTCCA (SEQ ID NO: 7), R-AGCACCAGAAACACGTGCACT (SEQ ID NO: 8); GAPDH: F-CTCTGCTCCTCCTGTTCGAC (SEQ ID NO: 9), R-GCGCCCCACCAAGCTCAAGA (SEQ ID NO: 10)). Fold increase was calculated by using the ΔΔCt method over non-infected untreated Huh-7.

### Viral Infectivity Assay

384-well plates (Perkin Elmer, 6057300) were seeded with Huh-7 cells at 3000 cells/well and allowed to adhere overnight. Compounds were then added to the cells and incubated for 4 hours. The plates were then transferred to BSL3 containment and infected with SARS-CoV-2 WA1 at a multiplicity of infection (MOI) of 0.2 in a 10 μL addition with shaking to distribute virus. After one hour of absorption, the virus inoculum was removed and media replaced with fresh compound. Uninfected cells and vehicle-treated cells were included as positive and negative control, respectively. Two days post-infection, cells were fixed with 4% PFA for 30 minutes at room temperature, permeabilized with 0.3% Triton X-100 and blocked with antibody buffer (1.5% BSA, 1% goat serum and 0.0025% Tween 20). The plates were then sealed, surface decontaminated, and transferred to BSL2 for staining with the optimized fluorescent dye-set: anti-nucleocapsid SARS-CoV-2 antibody (Antibodies Online, Cat #ABIN6952432) overnight treatment at 4C followed by staining with secondary antibody Alexa-647 (goat anti-mouse, Thermo Fisher, A21235), Hoechst-33342 Pentahydrate (bis-Benzimide) for nuclei staining (Thermo FIsher, H1398), HCS LipidTOX™ Green Neutral Lipid Stain (Thermo Fisher, H34475), and HCS CellMask™ Orange for cell delineation (Thermo Fisher H32713). iPSC (Boston University SPC2-ST-B2) derived alveolar epithelial cells (iAEC) maintained in 31) culture were dissociated to single cells and seeded in collagen coated 384-well plates at a seeding density of 8000 cells/well and grown to confluence over 72 hours. The following infection, compound treatment, and fixing was identical to that of Huh-7. Staining protocol for the iPSC-derived alveolar epithelial cells differed slightly with the addition of an anti-acetylated tubulin primary antibody (Cell Signaling, 5335), instead of HCS CellMask Orange, and the use of an additional secondary Alexa 488 (donkey anti-rabbit, Jackson ImmunoResearch, 711-545-152).

### Compound Library

The compound library deployed for drug screening was created using the FDA-Approved Drugs Screening Library (Item No. 23538) from Cayman Chemical Company. This library of 875 compounds was supplemented with additional FDA approved drugs and rationally included clinical candidates from other vendors including MedChemExpress, Sigma Aldrich, and Tocris. The library was formatted in five 384-well compound plates and was dissolved in DMSO at 10 mM. Hololactoferrin (Sigma Aldrich, L4765), apolactoferrin (Jarrow Formulas, 121011) and transferrin (Sigma Aldrich, T2036) were handled separately and added manually in cell culture media. Dilution plates were generated for qHTS at concentrations of 2 mM, 1 mM, 500 μM, 250 μM and 50 μM.

qHTS Primary Screen and Dose Response Confirmation

For the qHTS screen, compounds were added to cells using a 50 nL pin tool Caliper Life Sciences Sciclone ALH 3000 Advanced Liquid Handling system at the University of Michigan Center for Chemical Genomics (CCG). Concentrations of 2 μM, 1 μM, 500 nM, 250 nM and 50 nM were included for the primary screen. Post qHTS screen, all compounds were dispensed using an HP D300e Digital Compound Dispenser and normalized to a final DMSO concentration of 0.1% DMSO. Confirmation dose response was performed in triplicate and in 10-point:2-fold dilution.

### Imaging

Stained cell plates were imaged on both Yokogawa CQ1 and Thermo Fisher CX5 high content microscopes with a 20×/0.45 NA LUCPlan FLN objective. Yokogawa CQ1 imaging was performed with four excitation laser lines (405 nm/488 nm/561 nm/640 nm) with spinning disc confocal and 100 ms exposure times. Laser power was adjusted to yield optimal signal to noise ratio for each channel. Maximum intensity projection images were collected from 5 confocal planes with a 3 micron step size. Laser autofocus was performed and nine fields per well were imaged covering approximately 80% of the well area. The Thermofisher CX5 with LED excitation (386/23 nm, 485/20 nm, 560/25 nm, 650/13 nm) was also used and exposure times were optimized to maximize signal/background. Nine fields were collected at a single Z-plane as determined by image-based autofocus on the Hoechst channel. The primary qHTS screen was performed using CX5 images and all dose-response plates were imaged using the CQ1.

### Image Segmentation and Feature Extraction

The open source CellProfiler software was used in an Ubuntu Linux-based distributed Amazon AWS cloud implementation for segmentation, feature extraction and results were written to an Amazon RDS relational database using MySQL. A pipeline was developed to automatically identify the nuclei, cell, cytoplasm, nucleoli, neutral lipid droplets and syncytia for feature extraction. Multiple intensity features and radial distributions were measured for each object in each channel and cell size and shape features were measured. Nuclei were segmented using the Hoechst-33342 image and the whole cell mask was generated by expanding the nuclear mask to the edge of the Cell Mask Orange image.

### Data Pre-Processing

Cell level data were pre-processed and analyzed in the open source Knime analytics platform (Berthold et al. 2009). Cell-level data was imported into Knime from MySQL, drug treatment metadata was joined and features were centered and scaled. Features were pruned for low variance (<5%) and high correlation (>95%) and resulted in 660 features per cell.

### Machine Learning—Infectivity Score and Field-Level Scoring

Multiple logistic regression as implemented in the statistical language and environment R was used to identify features characteristic of cells within infected wells. Models were fit to cells from infected and uninfected control wells in the first five plate-series of the quantitative high throughput screen. As an independent benchmark, these logistic regression models were validated against a manually selected set of individual infected and uninfected cells; features which degraded performance on the benchmark were excluded from the model. The final model included only virus channel intensity features in the cell and cytoplasm ROIs. As a threshold for initial classification, the minimum value from virus-infected cells in the benchmark was used; the final decision rule is given in Eq. 1.

A cell is infected if(Cells_Intensity_IntegratedInensityEdge_Virus×0.1487025+Cells_Intensity_MeanIntensityEdge_Virus×−38,40196+Cells_Intensity_MaxIntensityEdge_Virus×42.70269+Cytoplasm_Intensity_StdIntensity_Virus×42.54849)≥1.525285 (Eq.1):

Then, individual field images from the infected control were categorized as confirmed-infected when the mean feature values, across all cells in the field, were above the threshold in Eq. 1. Using mean values for all 660 cell-profiler features in each field, a random forest classifier was trained to predict a probability of membership in the category of uninfected control fields vs confirmed-infected fields. The output of this random forest classifier is reported as “Probpos” (for the positive, uninfected control), throughout. Field level mean/median feature values were computed and a random forest model was fit between the positive control (32 uninfected wells) and the negative control (32 infected wells, 0.1% DMSO vehicle treated) with 80/20 cross validation. The compound treated wells were scored with the RF model and the efficacy score was normalized to the individual plate.

### UMAP Embedding

The embed_umap application of MPLeam (v0.1.0, https://github.com/momeara/MPLearn) was used to generate UMAP embeddings. Briefly, each for a set of cells, each feature was per-plate standardized and jointly orthogonalized using sklearn. IncrementalPCA(n_components=379, batch_size=1000). Then features were embedded into 2-dimensions using umap-learn (v0.4.1) (McInnes et al. 2018) with umap.UMAP(n_components=2, n neighbors=15, min_dist=0, init=‘spectral’, low_memory=True, verbose=True). Embeddings were visualized using Holovies Datashader (v1.12.7)(Stevens et al. 2015), using histogram equalization and the viridis color map.

### HC Stratominer

HC Stratominer (Core Life Analytics, Utrecht NL) (Core Life Analytics, Utrecht NL) was used as an independent method for hit-calling and performs fully automated/streamlined cell-level data pre-processing and score generation. IC Stratominer was also used to fit dose response curves for qHTS.

### ACAS

Compound registration and assay data registration were performed using the open source ACAS platform (Refactor BioSciences github https://github.com/RefactorBio/acas).

### Dose-Response Analysis and Compound Selection—in the Initial Screen Including all Unscheduled Small Molecule FDA Approved Drugs

In qHTS screening, a compound was selected to be carried forward into full dose response confirmation when meeting one of the following criteria: 1) Probpos greater than 0.75 for the median field in at least three concentrations, with per-field cell counts at least 60% of the positive control, and without an observed standard deviation in Probpos across-fields-in-the-well of 0.4 or greater, 2) a dose-response relationship with probpos was observed (by inspection) across the five concentrations tested, including compounds with Propbos greater than 0.90 at the two highest concentrations, or 3) compounds of interest not meeting this criteria were carried forward if reported positive in the literature or we being evaluated in clinical trials for COVID-19.

### Dose Response Analysis in the Confirmation and Combinatorial Screening

Due to the spatial inhomogeneity of infected cells across a single well, approximately half of the fields were undersaturated, leading to a consistent distribution in Probpos that saturates in the top third of 27 rank-ordered fields (from 9 fields and triplicate wells) for each concentration tested. The Probpos effect for a compound concentration was tabulated by averaging the top third of rank ordered fields. Outlier fields with high Probpos values were visually inspected and eliminated if artifacts (segmentation errors or debris) were observed. Cells treated with known fluorescence drugs including Clofazimine, were confirmed to not have spectral interference. Dose response curves were fit with Graphpad Prism using a semilog 4-parameter variable slope model.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. In addition, the following references referred to herein are incorporated by reference in their entireties:
1. Xiao, F. et al. Evidence for Gastrointestinal Infection of SARS-CoV-2. Gastroenterology (2020). doi:10.1053/j.gastro.2020.02.055
2. Lin, L. et al. Gastrointestinal symptoms of 95 cases with SARS-CoV-2 infection. Gut (2020). doi:10.1136/gutjnl-2020-321013
3. Avula, A. et al. COVID-19 presenting as stroke. Brain. Behav. Immun. (2020). doi:10.1016/j.bbi.2020.04.077
4. Kochi, A. N., Tagliari, A. P., Forleo, G. B., Fassini, G. M. & Tondo, C. Cardiac and arrhythmic complications in patients with COVID-19. Journal of Cardiovascular Electrophysiology (2020). doi:10.1111/jce.14479
5. Mulangu, S. et al. A randomized, controlled trial of Ebola virus disease therapeutics. N. Engl. J. Med. (2019). doi:10.1056/NEJMoa1910993
6. Oprea, T. I. et al. Drug repurposing from an academic perspective. Drug Discovery Today: Therapeutic Strategies (2011). doi:10.1016/j.ddstr.2011.10.002
7. Chu, H. et al. Comparative tropism, replication kinetics, and cell damage profiling of SARS-CoV-2 and SARS-CoV with implications for clinical manifestations, transmissibility, and laboratory studies of COVID-19: an observational study. The Lancet Microbe (2020). doi:10.1016/s2666-5247(20)30004-5
8. Hoffmann, M. et al. SARS-CoV-2 Cell Entry Depends on ACE2 and TMPRSS2 and Is Blocked by a Clinically Proven Protease Inhibitor. Cell (2020). doi:10.1016/j.cell.2020.02.052
9. Riva, L. et al. A Large-scale Drug Repositioning Survey for SARS-CoV-2 Antivirals. bioRxiv (2020). doi:10.1101/2020.04.16.044016
10. Katie Heiser et al. Identification of potential treatments for COVID-19 through artificial intelligence enabled phenomic analysis of human cells infected with SARS-CoV-2. bioRxiv (2020). doi:10.1101/2020.04.21.054387
11. Jeon, S. et al. Identification of antiviral drug candidates against SARS-CoV-2 from FDA-approved drugs. Antimicrob. Agents Chemother. (2020). doi:10.1128/AAC.00819-20
12. McInnes, L., Healy, J., Saul, N. & Großberger, L. UMAP: Uniform Manifold Approximation and Projection. J. Open Source Softw. (2018). doi:10.21105/joss.00861
13. Lang, J. et al. Inhibition of SARS pseudovirus cell entry by lactoferrin binding to heparan sulfate proteoglycans. PLoS One (2011). doi:10.1371/journal.pone.0023710
14. Siqueiros-Cendón, T. et al. Immunomodulatory effects of lactoferrin. Acta Pharmacologica Sinica (2014). doi:10.1038/aps.2013.200
15. Jacob, A. et al. Derivation of self-renewing lung alveolar epithelial type II cells from human pluripotent stem cells. Nat. Protoc. (2019). doi:10.1038/s41596-019-0220-0
16. Hurley, K. et al. Reconstructed Single-Cell Fate Trajectories Define Lineage Plasticity Windows during Differentiation of Human PSC-Derived Distal Lung Progenitors. Cell Stem Cell (2020). doi:10.1016/j.stem.2019.12.009
17. Yeni, P. Update on HAART in HIV. in Journal of Hepatology (2006). doi:10.1016/j.jhep.2005.11.021
18. Evans, W. E. Pharmacogenetics of Thiopurine S-Methyltransferase and Thiopurine Therapy. in Therapeutic Drug Monitoring (2004). doi:10.1097/00007691-200404000-00018
19. Arbiser, J. L. & Moschella, S. L. Clofazimine: A review of its medical uses and mechanisms of action. Journal of the American Academy of Dermatology (1995). doi:10.1016/0190-9622(95)90134-5
20. Alghamdi, S., Leoncikas, V., Plant, K. E. & Plant, N. J. Synergistic interaction between lipid-loading and doxorubicin exposure in Huh7 hepatoma cells results in enhanced cytotoxicity and cellular oxidative stress: Implications for acute and chronic care of obese cancer patients. Toxicol. Res. (Camb). (2015). doi:10.1039/c5tx00173k
21. Hibbs, A. M. & Lorch, S. A. Metoclopramide for the treatment of gastroesophageal reflux disease in infants: A systematic review. Pediatrics (2006). doi:10.1542/peds.2005-2664
22. Hajifathalian, K. et al. SARS-COV-2 infection (coronavirus disease 2019) for the gastrointestinal consultant. World J. Gastroenterol. (2020). doi:10.3748/wjg.v26.i14.1546
23. Roscow, O., Ganassin, R., Garver, K. & Polinski, M. Z-FA-FMK demonstrates differential inhibition of aquatic orthoreovirus (PRV), aquareovirus (CSRV), and rhabdovirus (IHNV) replication. Virus Res. (2018). doi:10.1016/j.virusres.2017.11.024
24. Ou, X. et al. Characterization of spike glycoprotein of SARS-CoV-2 on virus entry and its immune cross-reactivity with SARS-CoV. Nat. Commun. (2020). doi:10.1038/s41467-020-15562-9
25. Pardanani, A. et al. TG101209, a small molecule JAK2-selective kinase inhibitor potently inhibits myeloproliferative disorder-associated JAK2V617F and MPLW515L/K mutations. Leukemia (2007). doi:10.1038/sj.leu.2404750
26. Wu, D. & Yang, X. O. TH17 responses in cytokine storm of COVID-19: An emerging target of JAK2 inhibitor Fedratinib. J. Microbiol. Immunol. Infect. (2020). doi:10.1016/j.jmii.2020.03.005
27. Zhang, W. et al. The use of anti-inflammatory drugs in the treatment of people with severe coronavirus disease 2019 (COVID-19): The experience of clinical immunologists from China. Clinical Immunology (2020). doi:10.1016/j.clim.2020.108393
28. Stebbing, J. et al. COVID-19: combining antiviral and anti-inflammatory treatments. The Lancet Infectious Diseases (2020). doi:10.1016/S1473-3099(20)30132-8
29. Treatment of Moderate to Severe Coronavirus Disease (COVID-19) in Hospitalized Patients. (2020). Available at: https://clinicaltrials.gov/ct2/showNCT04321993.
30. Delprat, B., Crouzier, L., Su, T. P. & Maurice, T. At the Crossing of ER Stress and MAMs: A Key Role of Sigma-1 Receptor? in Advances in Experimental Medicine and Biology (2020). doi:10.1007/978-3-030-12457-1_28
31. Friesland, M., Mingorance, L., Chung, J., Chisari, F. V. & Gastaminza, P. Sigma-1 Receptor Regulates Early Steps of Viral RNA Replication at the Onset of Hepatitis C Virus Infection. J. Virol. (2013). doi:10.1128/jvi.03557-12
32. Fung, T. S. & Liu, D. X. Coronavirus infection, ER stress, apoptosis and innate immunity. Frontiers in Microbiology (2014). doi:10.3389/fmicb.2014.00296
33. Moebius, F. F., Reiter, R. J., Hanner, M. & Glossmann, H. High affinity of sigma1-binding sites for sterol isomerization inhibitors: Evidence for a pharmacological relationship with the yeast sterol C8-C7 isomerase. Br. J. Pharmacol. (1997). doi:10.1038/sj.bjp.0701079
34. Diaz, J. L. et al. Synthesis and biological evaluation of the 1-arylpyrazole class of σ1 receptor antagonists: Identification of 4-{2-[5-methyl-1-(naphthalen-2-yl)-1H-pyrazol-3-yloxy]ethyl}morpholine (S1RA, E-52862). J. Med. Chem. (2012). doi:10.1021/jm3007323
35. Torres, V. et al. QT prolongation and the antiarrhythmic efficacy of amiodarone. J. Am. Coll. Cardiol. (1986). doi:10.1016/S0735-1097(86)80272-8
36. Vidal-Torres, A. et al. Effects of the selective sigma-1 receptor antagonist S1RA on formalin-induced pain behavior and neurotransmitter release in the spinal cord in rats. J. Neurochem. (2014). doi:10.1111/jnc.12648
37. Gris, G. et al. The selective sigma-1 receptor antagonist E-52862 attenuates neuropathic pain of different aetiology in rats. Sci. Rep. (2016). doi:10.1038/srep24591
38. Gordon, D. E. et al. A SARS-CoV-2 protein interaction map reveals targets for drug repurposing. Nature (2020). doi:10.1038/s41586-020-2286-9
39. Reghunathan, R. et al. Expression profile of immune response genes in patients with severe acute respiratory syndrome. BMC Immunol. (2005). doi:10.1186/1471-2172-6-2
40. Cutone, A. et al. Lactoferrin prevents LPS-induced decrease of the iron exporter ferroportin in human monocytes/macrophages. BioMetals (2014). doi:10.1007/s10534-014-9742-7
41. Conti, P. et al. Induction of pro-inflammatory cytokines (IL-1 and IL-6) and lung inflammation by COVID-19: anti-inflammatory strategies. Journal of biological regulators and homeostatic agents (2020). doi:10.23812/CONTI-E.
42. Lagunas-Rangel, F. A. & Chavez-Valencia, V. High IL-6/IFN-γ ratio could be associated with severe disease in COVID-19 patients. Journal of Medical Virology (2020). doi:10.1002/jmv.25900
43. Han, C. et al. Digestive Symptoms in COVID-19 Patients With Mild Disease Severity: Clinical Presentation, Stool Viral RNA Testing, and Outcomes. Am. J. Gastroenterol. (2020). doi:10.14309/ajg.0000000000000664
44. Oda, H. et al. Antiviral Effects of Bovine Lactoferrin on Human Norovirus. Biochem. Cell Biol. (2020). doi:10.1139/bcb-2020-0035
45. Mohamed, J. A. et al. A Novel Single-Nucleotide Polymorphism in the Lactoferrin Gene Is Associated with Susceptibility to Diarrhea in North American Travelers to Mexico. Clin. Infect. Dis. (2007). doi:10.1086/512199
46. Gu, J., Han, B. & Wang, J. COVID-19: Gastrointestinal Manifestations and Potential Fecal-Oral Transmission. Gastroenterology (2020). doi:10.1053/j.gastro.2020.02.054
47. Hung, I. F.-N. et al. Triple combination of interferon beta-1b, lopinavir-ritonavir, and ribavirin in the treatment of patients admitted to hospital with COVID-19: an open-label, randomised, phase 2 trial. Lancet (2020). doi:10.1016/s0140-6736(20)31042-4
48. Berthold, M. R. et al. KNIME: The konstanz information miner. in 4th International Industrial Simulation Conference 2006, ISC 2006 (2006). doi:10.1007/978-3-540-78246-9_38
49. Stevens, J.-L., Rudiger, P. & Bednar, J. HoloViews: Building Complex Visualizations Easily for Reproducible Science. in Proceedings of the 14th Python in Science Conference (2015). doi:10.25080/majora-7b98e3ed-00a

1. A method for treating, ameliorating and/or preventing a condition related to viral infection in a subject, comprising
administering to the subject a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, and niclosamide,
wherein the subject is a human subject suffering from or at risk of suffering from a condition related to viral infection.

2. The method of claim 1, wherein the condition related to viral infection is SARS-CoV-2 infection (e.g., COVID-19).

3-4. (canceled)

5. The method of claim 1, wherein the pharmaceutical composition comprises lactoferrin, wherein the lactoferrin is mammalian lactoferrin and/or recombinant lactoferrin.

6. The method of claim 1, wherein the administering is oral, topical or intravenous.

7. (canceled)

8. The method of claim 1,
wherein the amount of the pharmaceutical composition that is administered is about 1 mg to about 10 g per day, or
wherein the amount of the pharmaceutical composition that is administered is about 10 mg to about 1 g per day.

9. (canceled)

10. The method of claim 1, further comprising administering to the subject remdesivir and/or hydroxychloroquine.

11. The method of claim 1, wherein administration of the pharmaceutical composition results in one or more of suppression of pro-inflammatory cytokine activity within the subject, enhancement of NK cell activity within the subject, enhancement of neutrophil activity within the subject, and inhibition of viral entry into the subject's cells through inhibiting binding of the virus with heparin sulfate proteoglycan within such cells.

12-33. (canceled)

34. The method of claim 1, wherein the treating, ameliorating and/or preventing a condition related to viral infection in a subject comprises treating, ameliorating and/or preventing symptoms related to the viral infection, wherein the symptoms related to viral infection in a subject are one or more of fever, fatigue, dry cough, myalgias, dyspnea, acute respiratory distress syndrome, and pneumonia.

35-106. (canceled)

107. A method for inhibiting viral entry in a cell, comprising exposing the cell a pharmaceutical composition comprising one or more of lactoferrin, S1RA, entecavir, lomitapide, metoclopramide, bosutinib, thioguanine, fedratinib, Z-FA-FMK, amiodarone, verapamil, gilteritinib, clofazimine, and niclosamide.

108. The method of claim 107, wherein cell is at risk of viral infection.

109. The method of claim 107, wherein the cell has been exposed to a virus.

110. The method of claim 107, wherein the cell is in culture.

111. The method of claim 107, wherein the cell is a living cell in a subject.

112. The method of claim 107, wherein exposure of the cell to the pharmaceutical composition results in one or more of suppression of pro-inflammatory cytokine activity within the cell, enhancement of NK cell activity within the cell, enhancement of neutrophil activity within the cell, and inhibition of viral entry into the cell through inhibiting binding of the virus with heparin sulfate proteoglycan within the cell.

113-114. (canceled)

115. The method of claim 107, wherein cell is at risk of SARS-CoV-2 infection.

116. The method of claim 107, wherein the cell has been exposed to a SARS-CoV-2.

117. The method of claim 107, wherein the cell is a living cell in a human subject suffering from or at risk of suffering from COVID-19.

118. The method of claim 107, wherein exposure of the cell to the pharmaceutical composition results in suppression of IL-6 activity within the cell.

119. The method of claim 1, wherein administration of the pharmaceutical composition results in one or more of suppression of IL-6 activity.


SEQUENCE LISTING (10 sequences)

SEQ ID NO: 1 - DNA, 24 bp, Artificial sequence
  synthetic
ttgacatccctgaggagattaagc

SEQ ID NO: 2 - DNA, 21 bp, Artificial sequence
  synthetic
tcccacgtactccaacttcca

SEQ ID NO: 3 - DNA, 20 bp, Artificial sequence
  synthetic
ccagctgctgcatcccaccc

SEQ ID NO: 4 - DNA, 20 bp, Artificial sequence
  synthetic
aggggcgcaccttctcctca

SEQ ID NO: 5 - DNA, 16 bp, Artificial sequence
  synthetic
tggcgggcaacgaatt

SEQ ID NO: 6 - DNA, 18 bp, Artificial sequence
  synthetic
gggtgatctgcgccttca

SEQ ID NO: 7 - DNA, 21 bp, Artificial sequence
  synthetic
tcccacgtactccaacttcca

SEQ ID NO: 8 - DNA, 21 bp, Artificial sequence
  synthetic
agcaccagaaacacgtgcact

SEQ ID NO: 9 - DNA, 20 bp, Artificial sequence
  synthetic
ctctgctcctcctgttcgac

SEQ ID NO: 10 - DNA, 20 bp, Artificial sequence
  synthetic
gcgccccaccaagctcaaga